(19) United States
(12) Patent Application Publication
(10) Pub. No.: US 2011/0075797 A1
(43) Pub. Date: Mar. 31, 2011

(54) DEVICE FOR A COMPUTER TOMOGRAPHY GANTRY FOR TRANSFERING CONTACTLESSLY ELECTRICAL ENERGY

(75) Inventors: Christoph Loef, Aachen (DE); Peter Luerkens, Aachen (DE); Bernd Ackermann, Aachen (DE)

(73) Assignee: KONINKLIJKE PHILIPS ELECTRONICS N.V., Eindhoven (NL)

(21) Appl. No.: 12/994,481

(22) PCT Filed: May 26, 2009
(86) PCT No.: PCT/IB09/52188

§ 371 Date: Nov. 24, 2010

(30) Foreign Application Priority Data
Jun. 2, 2008 (EP) 08104200.4

(51) Int. Cl. A61B 6/00; H05G 1/10; H01F 38/18

(52) U.S. Cl. 378/15; 378/111; 336/118

(57) ABSTRACT

The invention provides a device for a computer tomography gantry (91) for transferring contactlessly electrical energy from a stationary part of the gantry (92) to a rotary part of the gantry (93), wherein the device comprises a first power transformer, a second power transformer, wherein the first and the second power transformers are adapted for transferring the electrical energy, wherein the first power transformer comprises a first winding (506, 507, 542, 602, 601, 1202, 1301, 1401) out of the group consisting of a first set of primary windings and a first set of secondary windings of the first power transformer, wherein the second power transformer comprises a second winding (508, 509, 543, 603, 604, 1204, 1302, 1402) out of the group consisting of a second set of primary windings and a second set of secondary windings of the second power transformer, wherein the first set of primary windings and the second set of primary windings being adapted to be mounted on the stationary part of the gantry, wherein the first set of secondary windings and the second set of secondary windings being adapted to be mounted on the rotary part of the gantry (93), wherein the device is adapted to balance the currents of the first winding and the second winding. A further aspect of the invention is a computer tomography gantry (91) comprising a device according to the inventive concept. Furthermore, it is an aspect of the invention a method for transferring contactlessly electrical energy from a stationary part of a gantry (92) to a rotary part of a gantry (93) comprising the steps of balancing currents with the help of a device according to the invention.

### FIELD OF THE INVENTION

The present invention relates to a device for a computer tomography gantry for transferring contactlessly electrical energy from a stationary part of the gantry to a rotary part of the gantry and a computer tomography gantry comprising such a device and a method for transferring contactlessly electrical energy from a stationary part of a computer tomography gantry to a rotary part of the gantry.

### BACKGROUND OF THE INVENTION

Usually, power transformers of computer tomography gantries are operated with high frequency. The high frequency operation renders the possibilty to reduce size and weight of the energy storing devices (capacitors, inductors, transformers) used in the system. Usually, E-cores are used for the transformers in order to avoid external leakage flux. Thus, a long winding path first clockwise and then counterclockwise along the circumference will cause high values of inductances. Using a resonant converter system, the resulting leakage inductance of the transformer must be low in order to transmit the required power level. When high power transmissions for the rotary part of the gantry is required, a plurality of inverters will be used. In this case, each of the inverters generates a fraction of the totally required power to transfer to the rotary part of the gantry. With respect to manufacturing tolerances and temperature influences, the components of the inverters as well as the characteristics of the transformers are different. Thus, the fractions of power, which are transferred by the different inverters are not equal. This leads to an unequal workload of the different inverters. As a result thereof cogging forces will occur and thus the rotary part of the gantry could be bent during rotation of the rotary part of the gantry.

### SUMMARY OF THE INVENTION

It would be desirable to provide an improved device for balancing the workload of the different inverters, which supply the power transformer. As a result thereof cogging forces and bending of the rotary part of the gantry would be avoided. This would lead to a longer lifetime of the power transformer of the computer tomography gantry and the gantry itself.

The invention provides a device for a computer tomography gantry for transferring contactlessly electrical energy from a stationary part of the gantry to a rotary part of the gantry, wherein the device comprises a first power transformer, a second power transformer, wherein the first and the second power transformers are adapted for transferring the electrical energy, wherein the first power transformer comprises a first winding out of the group consisting of a first set of primary windings and a first set of secondary windings of the first power transformer, wherein the second power transformer comprises a second winding out of the group consisting of a second set of primary windings and a second set of secondary windings of the second power transformer, wherein the first set of primary windings and the second set of primary windings being adapted to be mounted on the stationary part of the gantry, wherein the first set of secondary windings and the second set of secondary windings being adapted to be mounted on the rotary part of the gantry, wherein the device is adapted to balance the currents of the first winding and the second winding.

The balancing of the different currents of the single windings is important with respect to the aim of a equally rotation without fluctuations. Fluctuations of the rotation would lead to uncontrollable vibrations. In a worst case scenario these vibrations could lead to damages to the computer tomography gantry.

The invention provides also a computer tomography gantry comprising a device according to one of the claims 1 to 13.

Further, the invention provides a method for transferring contactlessly electrical energy from a stationary part of a computer tomography gantry to a rotary part of the gantry, comprising the steps of balancing currents with the help of a device according to one of the claims 1 to 13.

Further embodiments are incorporated in the dependent claims.

According to the present invention it is provided a device, wherein the first winding and the second winding are magnetically coupled in such a way that the device is adapted to balance the currents of the first winding and the second winding.

This arrangement balances the currents without requiring additional components such as an additional current compensating choke. It is only necessary to couple the magnetic relevant areas of windings with different currents. The common magnetic flux would result in a balancing of the different currents.

According to an exemplary embodiment it is provided a device, further comprising a current balancing transformer, which is arranged in such a way so that being adapted for balancing the currents of the first winding and the second winding. The term current balancing transformer corresponds to the term current balancing choke. The current balancing choke is a special variation of a transformer.

It is also possible to arrange discrete elements to balance the currents of the different windings. These elements generate an additional magnetic coupling between the different windings. This magnetic coupling leads to the balance of the currents. This embodiment is advantageously because of the fact that a special arrangement of the windings of the transformer is not necessary. Further, the additional element, the current balancing choke/transformer are available in every size and requirements.

According to the present invention it is provided a device, wherein the first winding is a first primary winding of the first power transformer and the second winding is a second primary winding of the second power transformer, so that the device is adapted for balancing the currents of the first primary winding and the second primary winding.

According to an exemplary embodiment it is provided a device, wherein the first winding is a first secondary winding of the first power transformer and the second winding is a second secondary winding of the second power transformer, so that the device is adapted for balancing the currents of the first secondary winding and the second secondary winding.

According to an exemplary embodiment it is provided a device, wherein the first and the second power transformers are adapted to be operated with currents of a high frequency, such that the power transformers are adapted to transfer energy in a high frequency.

In order to transfer the immense electrical energy, which is required from the components on the rotary part of the gantry, it is necessary to use a high frequency application. Therefore, it is advantageously to adapt the device according to the inventive concept to the requirements of a high frequency application.

According to an exemplary embodiment it is provided a device, further comprising an inverter, wherein the inverter is adapted to be connected with the first and the second power transformer such that the inverter feeds the first and the second power transformer with electrical energy.

According to an exemplary embodiment it is provided a device, further comprising a rectifier, wherein the rectifier is adapted to be connected with the first and the second power transformer such that the rectifier rectifies the output voltage of the first and the second power transformer.

Especially, the problem of current balancing is on hand in case of only one single inverter, which supplies the primary windings, or in case of only one single rectifier, which rectifies the voltages at the secondary side of the transformer. In both cases there is no possibility to adjust the different currents, because the inverter/rectifier can only influence the common current of all windings of the primary or the secondary side of the transformer. Therefore, the solution provided by this invention is especially advantageously with respect to the above-mentioned situations.

According to an exemplary embodiment it is provided a device, wherein the device further comprises a third power transformer, a fourth power transformer, wherein the first power transformer is adapted to be supplied by a first inverter, wherein the second power transformer is adapted to be supplied by a second inverter, wherein the third power transformer is adapted to be supplied by a third inverter, wherein the fourth power transformer is adapted to be supplied by a fourth inverter, wherein the first inverter is arranged close to the second inverter, wherein the third inverter is arranged close to the fourth inverter, wherein the first inverter is supplied by a mains input stage via a first supply line, wherein the second inverter is supplied by the mains input stage via a second supply line, wherein the third inverter is supplied by the mains input stage via a third supply line, wherein the fourth inverter is supplied by the mains input stage via a fourth supply line, wherein the first supply line is considerably shorter than the second supply line, wherein the third supply line is considerably shorter than the fourth supply line.

According to another exemplary embodiment it is provided a device, wherein a winding out of a group consisting of the first set of primary windings and the first set of secondary windings of the first power transformer and the second set of primary windings and the second set of secondary windings of the second power transformer is arranged in a circular arc.

According to another exemplary embodiment it is provided a device, comprising a first power transformer with a first winding out of a group consisting of the first set of primary windings and the first set of secondary windings, a second power transformer with a second winding out of a group consisting of the second set of primary windings and the second set of secondary windings, a third power transformer with a third winding out of a group consisting of the third set of primary windings and the third set of secondary windings, a fourth power transformer with a fourth winding out of a group consisting of the fourth set of primary windings and the fourth set of secondary windings, wherein the first, the second, the third and the fourth windings are arranged in four circular arcs.

According to another exemplary embodiment it is provided a device, comprising a first power transformer with a first primary winding, a second power transformer with a second primary winding, a third power transformer with a third primary winding, a fourth power transformer with a fourth primary winding, a first current balancing transformer, wherein a winding of the first current balancing transformer is wound around a part of the first primary winding and around a part of the second primary winding, so that the first current balancing transformer is adapted for balancing the currents of the first and second primary windings,a second current balancing transformer, wherein a winding of the second current balancing transformer is wound around a part of the second primary winding and around a part of the third primary winding, so that the current balancing transformer is adapted for balancing the currents of the second and third primary windings,a third current balancing transformer, wherein a winding of the third current balancing transformer is wound around a part of the third primary winding and around a part of the fourth primary winding, so that the current balancing transformer is adapted for balancing the currents of the third and fourth primary windings.

According to another exemplary embodiment it is provided a device, comprising a first power transformer with a first secondary winding, a second power transformer with a second secondary winding, a third power transformer with a third secondary winding, a fourth power transformer with a fourth secondary winding, a first current balancing transformer, wherein a winding of the first current balancing transformer is wound around a part of the first secondary winding and around a part of the second secondary winding, so that the first current balancing transformer is adapted for balancing the currents of the first and second secondary windings,a second current balancing transformer, wherein a winding of the second current balancing transformer is wound around a part of the second secondary winding and around a part of the third secondary winding, so that the current balancing transformer is adapted for balancing the currents of the second and third secondary windings, and a third current balancing transformer, wherein a winding of the third current balancing transformer is wound around a part of the third secondary winding and around a part of the fourth secondary winding, so that the current balancing transformer is adapted for balancing the currents of the third and fourth secondary windings.

It may be seen as a gist of the present invention to provide a possibilty to balance currents, which are supplied by inverters to windings of transformers. The corresponding windings of the transformer can be primary windings or secondary windings or both (the primary windings and the secondary windings can be balanced). This leads to the result that the workload for different inverters/windings are equal. Therefore, asymmetrical workload is avoided, which results in the prevention of bending of the computer tomography gantry.

It should be noted that the above features may also be combined. The combination of the above features may also lead to synergetic effects, even if not explicitly described in detail.

These and other aspects of the present invention will become apparent from and elucidated with reference to the embodiments described hereinafter.

### BRIEF DESCRIPTION OF THE DRAWINGS

Exemplary embodiments of the present invention will be described in the following with reference to the following drawings.

FIG. 1 shows a part of a computer tomography gantry,

FIG. 2 shows a part of a transformer,

FIG. 3 shows a part of a transformer with four inverters,

FIG. 4 shows a part of a transformer with four inverters,

FIG. 5 shows a part of a transformer with current balancing chokes,

FIG. 5A shows a part of a transformer with four inverters,

FIG. 5B shows a part of a transformer with two current balancing chokes,

FIG. 5C shows a part of a transformer with three current balancing chokes,

FIG. 6 shows a part of a transformer with three current balancing chokes,

FIG. 7 shows a diagram of a part of a computer tomography gantry,

FIG. 8 shows a part of a transformer,

FIG. 9 shows a part of a transformer,

FIG. 10 shows rectifiers,

FIG. 11 shows a part of a transformer,

FIG. 12 shows a part of a transformer with six rectifiers,

FIG. 13 shows a part of a transformer,

FIG. 14 shows a part of a transformer,

FIG. 15 shows a computer tomography gantry.

### DETAILED DESCRIPTION OF EMBODIMENTS

The system described herein focuses on a system for contactlessly energy transmission, which provides energy transfer to a rotating dish, i.e. a rotating part of a computer tomography gantry. Further, an arrangement of windings is described, which provides the balancing of currents in different windings.

In this invention a rotary transformer is shown, which provides the use of high frequency operation and minimized amount of magnetic material. Further, it is avantageously that the losses in the windings according to the inventive concept will be reduced, especially with high frequencies. The major problems which are solved by this invention is an unequal flux distribution along the circumference of the rotary transformer and an unequal power transmission when the power transformer of the gantry comprises a plurality of primary or secondary windings.

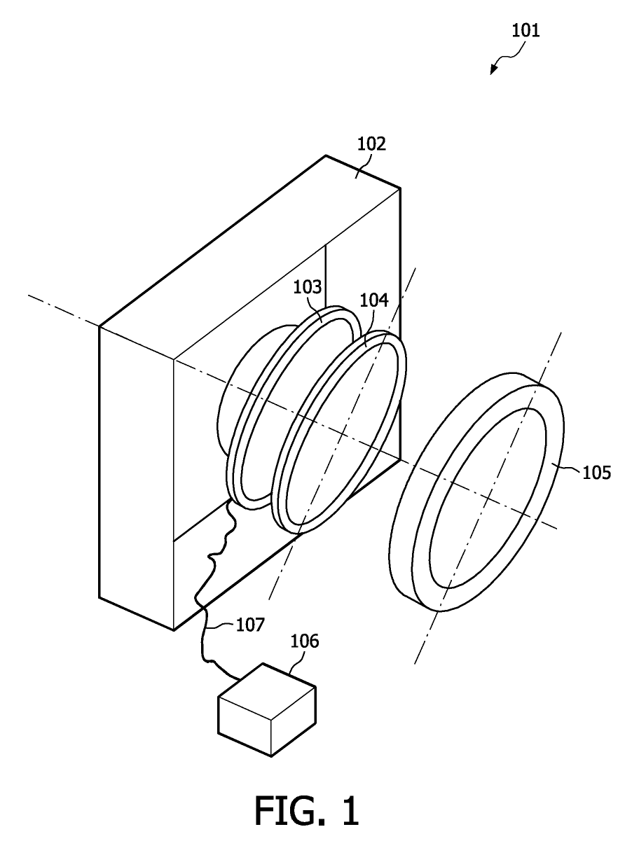

FIG. 1 shows a part of a computer tomography gantry 101, with a stationary part 102. It is depicted two stationary parts of the transformer 103, 104 and a rotary part of the transformer 105. The stationary parts of the transformer 103, 104 are supplied by an inverter 106 with the help of a supply line 107.

FIG. 1 shows essential elements of a computer tomography gantry 101, wherein a contactless energy transmission from the stationary part of the gantry 102 to the rotary part of the gantry 105 is applied. The system 101 consists of a frame 102 and a rotating part of the gantry 105, wherein the rotary part of the gantry is mounted by bearings. The primary side of a power transformer 103, 104 is arranged at the stationary part of the gantry 102. The secondary part of the transformer 105 is arranged at the stationary part of the gantry. The power transformer is used to transfer electrical energy from the stationary part of the gantry to the rotary part of the gantry. There can be also an auxiliary transformer, which is arranged in a similar way, to transmit electrical energy for auxiliary units, which are located on the rotating part of the gantry. It is also possible to arrange several power transformers to transmit electrical energy.

It is depicted only one inverter 106, which supplies the electrical energy to the gantry. It is also possible to use a plurality of inverters 106, in order to distribute the workload on several inverters 106. In this case the inverters 106 can be equipped with smaller, cheaper electronical elements.

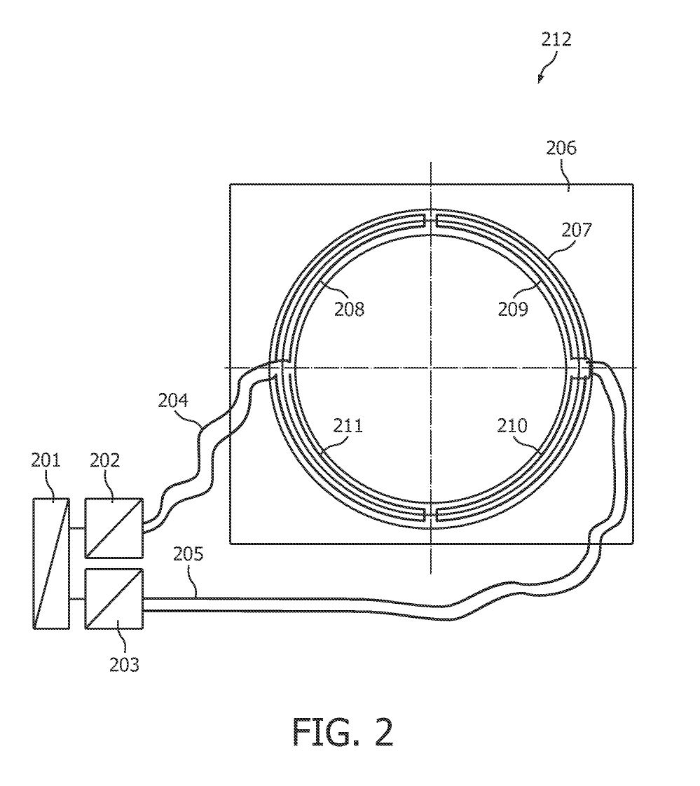

FIG. 2 shows a part of a transformer 212 with windings 208, 209, 210 and 211. The part of the transformer 212 belongs to the stationary part of the transformer, which is connected with the stationary part of the gantry 206. The different primary windings 208, 209, 210 and 211 of the transformer 212 are supplied by two inverters 202, 203, wherein a mains supply unit 201 feeds the two inverters 202 and 203. The four different windings 208, 209, 210 and 211 are supplied with the help of supply lines 204 and 205. The four different windings 208, 209, 210 and 211 are adapted to induce a magnetic flux in the core 207. The core 207 is usually E-shaped.

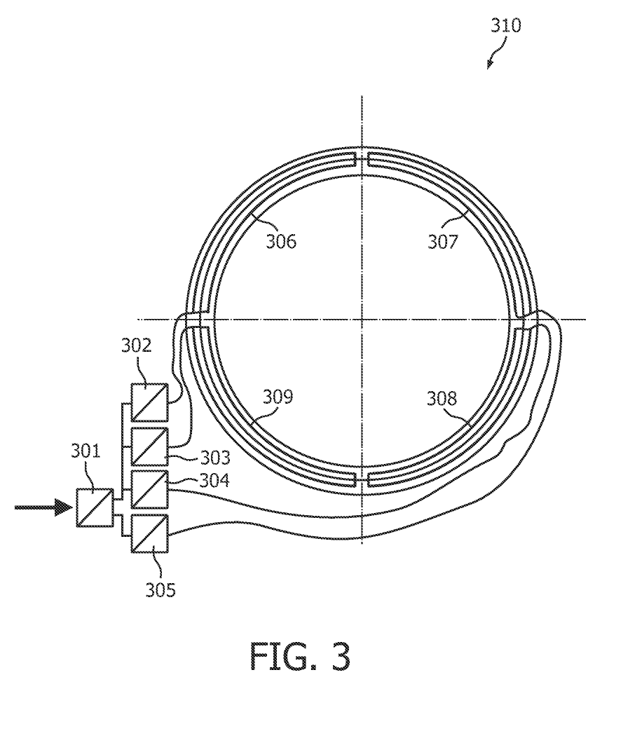

FIG. 3 shows a part of a transformer, wherein the part of the transformer is the stationary part of the transformer, which is connected with the stationary part of the gantry 310. The four different primary windings 306, 307, 308 and 309 are supplied by four different inverters 302, 303, 304 and 305. These four inverters 302, 303, 304 and 305 will be fed by a mains input stage 301. In this case every different winding 306, 307, 308 and 309 is supplied by a different inverter 302, 303, 304 and 305. Every different inverter 302, 303, 304 and 305 enables an user to adjust the currents to the different physical characteristics of the primary windings 306, 307, 308 and 309. According to this it is possible to supply the different primary windings 306, 307, 308 and 309 with the same currents, because the four different currents for the four different windings 306, 307, 308 and 309 can be adjusted separately.

FIG. 3 depicts four inverters 302, 303, 304, 305. Typically the inverters 302, 303, 304, 305 are located around the circumference of the power transformer. Other numbers of inverters are also realizable.

Due to mechanical tolerances the inductances of each winding 306, 307, 308, 309 on the primary side of the contactless power transformer will be different. Thus, the current in each winding 306, 307, 308, 309 and the flux induced by each winding 306, 307, 308, 309 will be different. This will cause unequal power transmission to the stationary side of the gantry during rotation of the rotary part of the gantry. Furthermore, cogging forces will occur and thus the rotating part of the gantry could be bent during rotation.

To overcome the bending of the rotating part of the gantry and to achieve identical current distribution in the primary windings 306, 307, 308, 309, the current in each winding 306, 307, 308, 309 must be identical. In case the currents in each winding 306, 307, 308, 309 is identical the flux induced by the windings 306, 307, 308, 309 will be identical at each rotational angle. Identical currents can be achieved with the help of current balancing chokes.

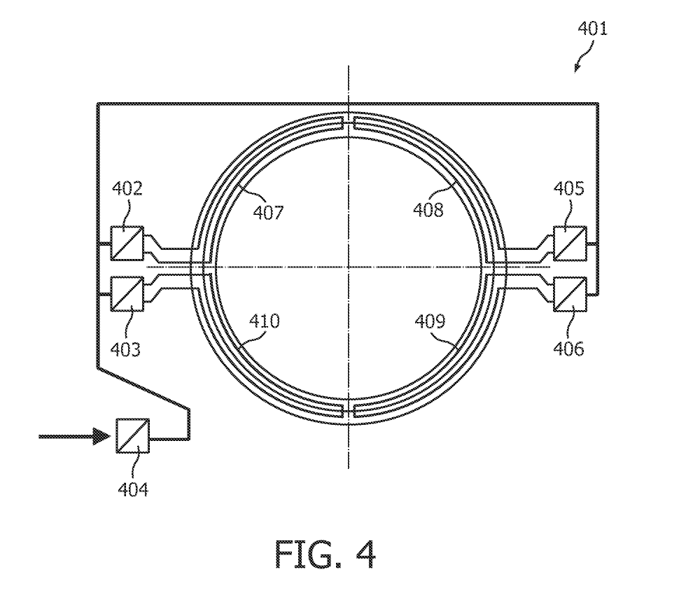

FIG. 4 shows a part of the stationary part of the transformer 401. The different four primary windings 407, 408, 409 and 410 are supplied by four different inverters 402, 403, 405 and 406. These four inverters 402, 403, 405, 406 are fed by a mains input stage 404.

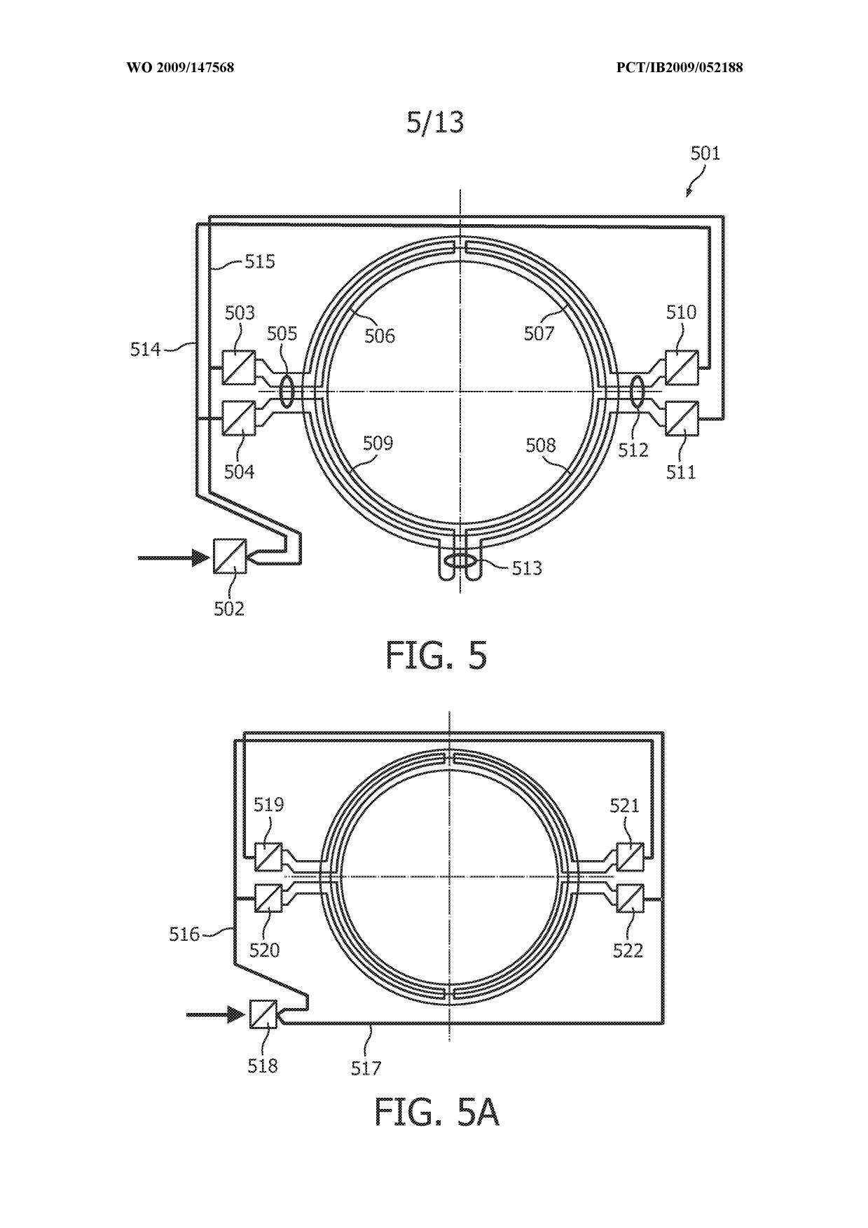

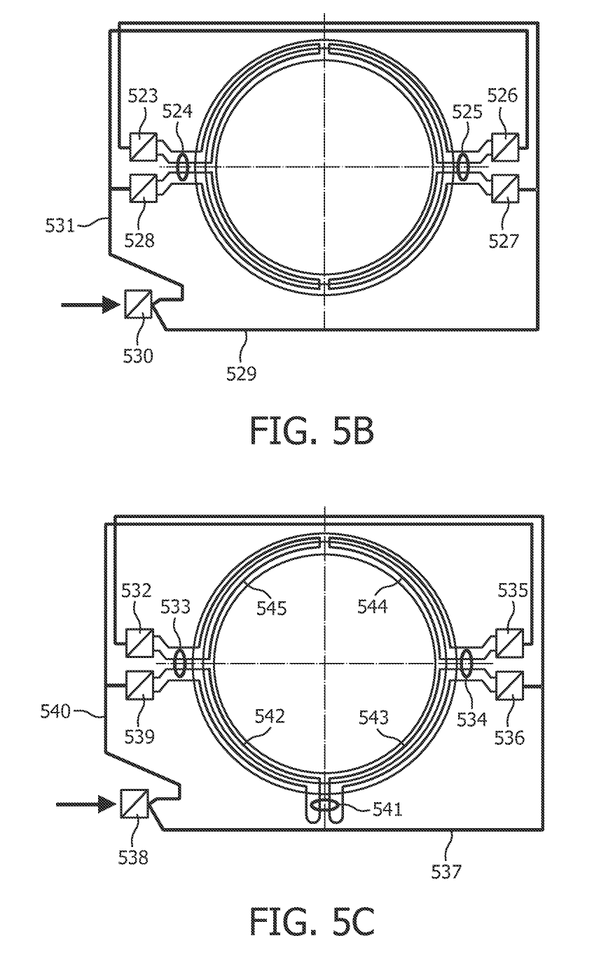

FIG. 5 shows a part of a transformer 501. It is shown four different primary windings 506, 507, 508 and 509. The primary winding 506 is supplied by the inverter 503, the primary winding 507 is supplied by the inverter 510, the primary winding 508 is supplied by the inverter 511, the primary winding 509 is supplied by the inverter 504. The inverters 503, 504, 510 and 511 are fed by a mains input stage 502. The four inverters 503, 504, 510 and 511 are supplied by supply lines 514 and 515. The arrangement of the supply lines 514 and 515 is such that the part of the supply line 515 which leads to the inverter 504 also supplies the inverter 510. The supply line 515 supplies the inverter 503 as well as the inverter 511. The currents of the inverter 503 and the inverter 504 will be balanced by a current balancing choke 505, such as the currents which are in the primary windings 506 and 509 are equal. The current balancing choke 505 is an additional discrete separate element, which has to be added. The currents of the inverters 510 and 511 by which the primary windings 507 and 508 are supplied will be balanced by a current balancing choke 512. As a result thereof, the currents of the primary windings 507 and 508 are equal. In order to adjust all four currents in the primary windings 506, 507, 508 and 509, it is necessary to insert a third current balancing choke 513. In order to enabling the addition of a third current balancing choke 513, it is also necessary to arrange two primary windings 508, 509 or the primary windings 506, 507 in a special way. It is also possible to balance the currents of the stationary part of the transformer by balancing the currents of the primary windings 506 and 507. A third possibility would be to balance the currents of the primary windings 506 and 508 and a fourth possibility would be the balancing of the currents 507 and 509.

The FIGS. 5A, 5B and 5C show also the same arrangement of four different primary windings and four different inverters which supply the four different primary windings.

FIG. 5A differs from the FIG. 5 by the different arrangement of supply lines 516 and 517. In FIG. 5A the supply line 516 supplies the inverter 520 and the inverter 521. This is the same arrangement as in FIG. 5 according to the supply line 514. The difference is the arrangement of the supply line 517. In FIG. 5A the supply line 517 supplies at first the inverter 522 and then the inverter 519. The difference to the FIG. 5 is that the supply line 517 does not run at first to the inverter 519 and then to the inverter 522. According to this the length of the part of the supply line 516 which leads to the inverter 520 is considerably short in comparison with the part of the supply line 516 which leads to the inverter 521. The length of the part of the supply line 517, which leads to the inverter 522 is short in comparison with the length of the part of the supply line 517, which leads to the inverter 522. Therefore, as a result thereof, the length of the supply lines to the inverter 519 and 520 are considerably different. The same for the pair of inverters 521, 522, wherein the length of the supply lines 516, 517 are considerably different also.

FIG. 5B shows the same arrangement of supply lines and inverters. With respect to the fact, that along a supply line 529, 531 there is a voltage decrease it is obvious, that the pairs of inverters 523, 528 as well as the pair of inverter 526 and 527 are supplied with considerably different voltages due to the fact that the length of supply lines are considerably different. Therefore, the currents which will be fed to the primary windings of the pairs of inverters are also different because of the voltage decrease along the supply lines, which are considerably different. Therefore, it is a considerable difference of the currents of the pair of inverters 523, 528 as well as the pair of the inverter 526, 527. Therefore, two points of considerably difference of currents are enforced at the output of the pairs of inverters 523, 528 and 526, 527. Therefore, the current balancing choke 524 as well as the current balancing choke 525 are placed at very useful sites in the arrangement of the primary windings and their feeding inverters. Resulting from the both current balancing chokes at very favourable sites of the arrangement, the difference of the currents of the pairs of the two primary windings is not considerably different. This leads to the fact, that a third current balancing choke (as described above in FIG. 5) can be omitted.

The FIG. 5C shows a part of the computer tomography gantry, wherein the mains input stage 538 supplies the inverters 532, 539, 535 and 536 in the same manner as in the arrangement of the FIG. 5B. There are also two current balancing chokes 533 and 534. The difference of the FIG. 5C in comparison with the FIG. 5B is the arrangement of a third current balancing choke 541. The third current balancing choke 541 is arranged in order to balance the currents of the primary windings 542 and 543. In order to receive the same value of current in all four primary windings 542, 543, 544 and 545. It is also possible to balance the currents of the primary windings 542 and 544 to arrive at four identical currents in the four primary windings 542, 543, 544, 545. Another possibility with the same result would be the balancing of the currents of the primary windings 545 and 543.

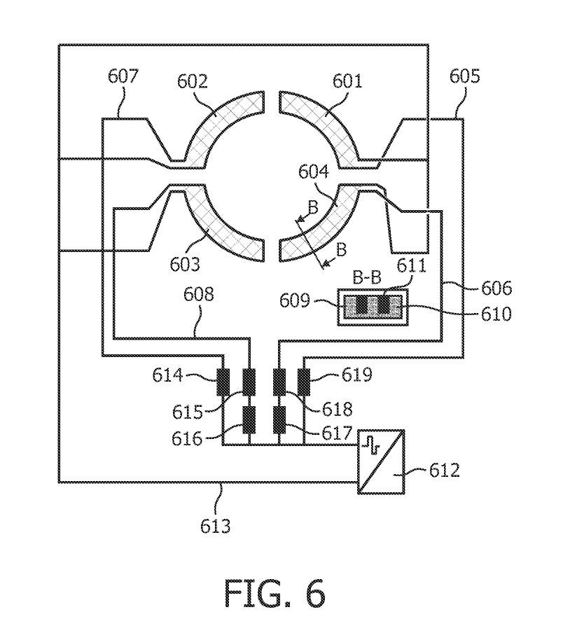

FIG. 6 shows an arrangement of four primary windings 601, 602, 603 and 604. The primary winding 602 is supplied by the supply line 607, the primary winding 601 is supplied by the supply line 605, the primary winding 603 is supplied by the supply line 608. The primary winding 604 is supplied by the supply line 606. All four supply lines 607, 608, 605 and 606 are fed by one single inverter 612. According to the fact that four primary windings 601, 602, 603 and 604 are supplied by one single inverter 612 it is not possible to adjust all four currents in the primary windings 601, 602, 603 and 604 such that the value of the currents are equal without further arrangements. The value of the currents of the different primary windings 601, 602, 603 and 604 depends on the physical characteristics of the primary windings 601, 602, 603 and 604. In order to adjust the currents of the four different primary windings 601, 602, 603 and 604 it is therefore necessary to balance the different currents. Therefore, the currents in the supply lines 607 and 608 are balanced by a current balancing choke which is realized by magnetic coupling with the help of the inductances 614 and 615. The currents in the supply lines 608 and 606 is balanced with the help of a current balancing choke, which is realized by the inductances 616 and 617. The currents in the supply lines 605 and 606 is balanced with the help of a current balancing choke which is realized by the inductances 618 and 619. With the help of the current balancing chokes 614, 615, 616, 617, 618 and 619 it is guaranteed that the four different primary windings 601, 602, 603 and 604 have the same value of current.

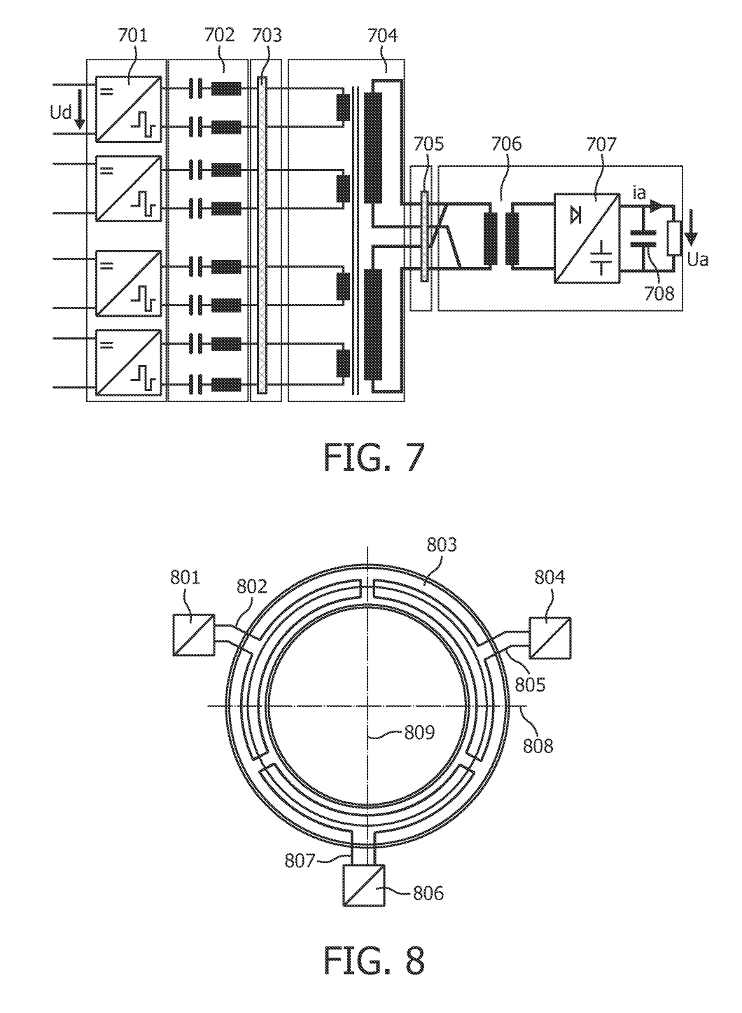

FIG. 7 shows the arrangement of current balancing chokes 705 at the secondary side of a power transformer. FIG. 7 shows schematically a part of a computer tomography gantry. It is shown an inverter 701, which transforms a DC voltage into a switched DC voltage. This switched voltage is supplied to a resonant circuit 702 with a capacitor and an inductance, wherein in this special case four different inverters 701 supply four different resonant circuits 702, which lead to four different currents which are fed to four different primary windings, which are galvanically isolated. In order to adjust the four different currents a current balancing choke 703 is implemented. The transformer 704 transforms the input voltage and supplies his output voltage to a further transformer 706, which is adapted to transform the voltage into a higher voltage. It is also implemented between the first transformer and the second transformer a current balancing choke 705 in order to adjust the two different currents at the secondary side of the transformer 704. The output voltage of the second transformer 706 is rectified and smoothened by an unit 707. The output of the unit 707 will be supplied to an unit on the rotary part of the computer tomography gantry, which is schematically depicted by a capacitor 708.

The current balancing chokes (transformers) 705 are located between the secondary windings of the first transformer and the successive rectifier 707 or a further transformer 706. If identical currents in all primary windings are achieved and identical currents in the secondary windings are achieved the transferred power and the resonance frequency are independent from the angular position of the power transformer.

FIG. 8 shows a part of the primary side of the transformer of a computer tomography gantry. The primary side of the transformer comprises three different primary windings 802, 805 and 807. The primary winding 802 is supplied by the inverter 801, the primary winding 805 is supplied by the inverter 804, the primary winding 807 is supplied by the inverter 806. The arrangement of the primary windings is symmetrically with respect to the center lines 809 and 808. It is also depicted the core 803 of the primary side of the transformer, wherein the core 803 has an E-shaped form.

Exemplarily, it is shown three inverters 801, 804, 806 at the primary side of the power transformer. Each of the inverters 801, 804, 806 is connected to a single primary winding 802, 805, 807. Each of the winding 802, 805, 807 covers a fraction of the core 803. Other arrangements with more primary windings or only two or only one winding are also realisable.

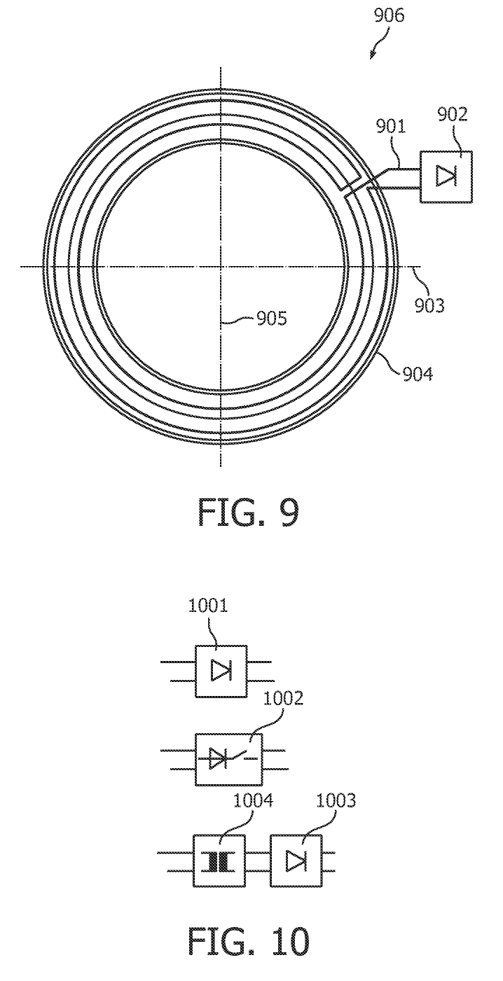

FIG. 9 shows a secondary part of a transformer for a computer tomography gantry 906. It is depicted a secondary part of the transformer with only one secondary winding 901, wherein the winding 901 is connected with a rectifier 902. The rectifier 902 rectifies an output voltage of the transformer. The arrangement is symmetrically with respect to the center lines 903 and 905. The secondary winding 901 is arranged in such a way that it can induce a magnetic flux in the core 904. The core 904 is E-shaped.

FIG. 10 shows three schematically diagrams of different electronic elements. The diagram 1001 represents an unit for rectifying. The element 1002 represents an unit which comprises diodes and switches. The element 1004 represents a transformer and the element 1003 represents a rectifier. FIG. 10 shows electronic loads, which can be connected to the secondary side of the power transformer. These loads can be used as alternatives to the rectifier, depicted in FIG. 9 (902).

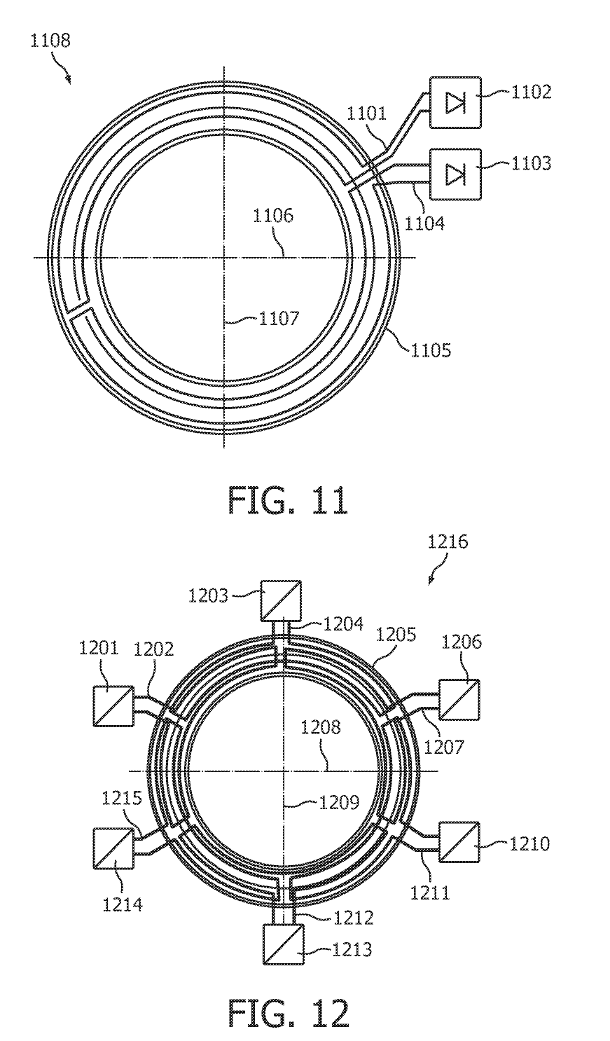

FIG. 11 shows the secondary side of a transformer 1108. The secondary side of the transformer 1108 comprises two different secondary windings 1101 and 1104. These two different windings 1101 and 1104 will be supplied to two different rectifiers 1102 and 1103. The arrangement is symmetrically with respect to the center lines 1106 and 1107. The secondary windings 1101 and 1104 are embedded in a core 1105. The core 1105 is typically designed in an E-shape form.

FIG. 12 shows a part of the primary side of a transformer 1216. It is depicted six different primary windings 1202, 1204, 1207, 1211, 1212 and 1215. The primary winding 1202 is supplied by an inverter 1201, the primary winding 1204 is supplied by the inverter 1203, the primary winding 1207 is supplied by the inverter 1206, the primary winding 1211 is supplied by the inverter 1210 and the primary winding 1212 is supplied by the inverter 1213. The six primary windings 1202, 1204, 1207, 1211, 1212 and 1215 can be galvanically isolated and have six different currents. In order to adjust the six different currents the six galvanically isolated primary windings are magnetically coupled with the help of a special arrangement. The primary winding 1202 is magnetically coupled with the primary winding 1204 and the primary winding 1214 with the help of a special arrangement, wherein a common magnetically flux results by overlapping windings. The primary winding 1204 is coupled with the primary winding 1207. The primary winding 1207 is coupled with the primary winding 1211. The primary winding 1211 is magnetically coupled with the primary winding 1212. The primary winding 1212 is magnetically coupled with the primary winding 1212. The primary winding 1212 is magnetically coupled with the primary winding 1215. With the help of the special arrangement of the primary windings 1202, 1204, 1207, 1211, 1212 and 1215 it is possible to adjust the different currents without the help of discrete, separated components such as current balancing chokes.

Due to mechanical tolerances the inductances of each of the winding 1202, 1204, 1207, 1211, 1212, 1215 around the circumference on the primary side of the contactless power transformer will be different. Thus, the current and the flux, which is induced by a winding 1202, 1204, 1207, 1211, 1212, 1215, in each segment will be different. This will cause unequal power transmitted to the secondary side of the power transformer during the rotation of the secondary part of the gantry. Furthermore, cogging forces will occur and thus the rotating gantry could be bent during rotation. To overcome the bending of the rotating part of the gantry and to achieve identical current distribution in the primary windings 1202, 1204, 1207, 1211, 1212, 1215 around the circumference, the current in each of the windings must be identical. When the current in each of the windings 1202, 1204, 1207, 1211, 1212, 1215 is identical the flux generated along the circumference will be identical at each rotational angle.

Exemplarily, FIG. 12 shows six inverters 1203, 1206, 1210, 1213, 1214, 1201, which feed six primary windings 1202, 1204, 1207, 1211, 1212, 1215. Other numbers of inverters are also realisable. According to the inventive concept neighbouring windings are overlapping in a symmetric way. The magnetic coupling between neighbouring windings in these overlap regions provides the functionality of current balancing chokes but without using additional separate discrete components.

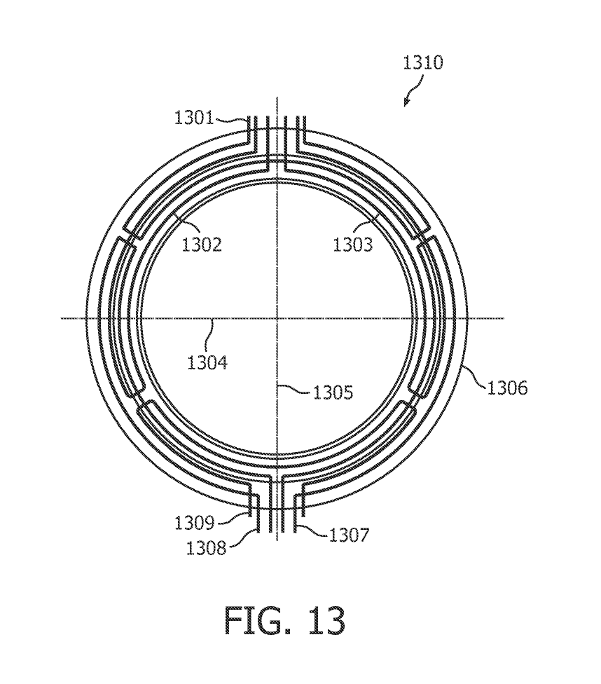

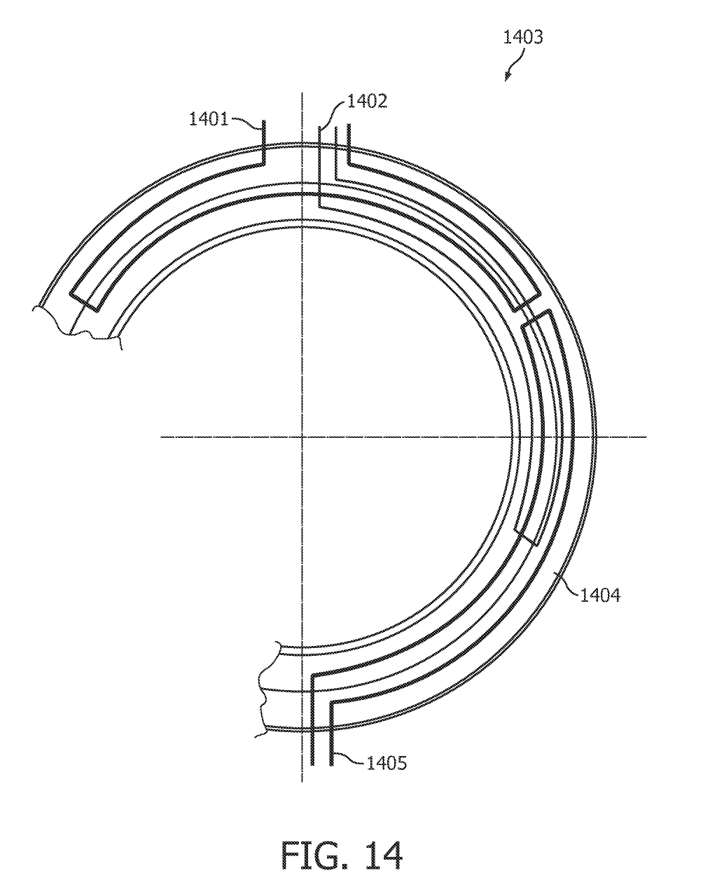

FIG. 13 shows a part of a transformer 1310. This part of a transformer could be a part of the primary side of the transformer as well as a part of the secondary side of the transformer. It is depicted six different galvanically isolated windings 1301, 1302, 1303, 1309, 1308 and 1307. The windings 1301, 1302, 1303, 1309, 1308 and 1307 are magnetically coupled in such a way that alone with the help of the special arrangement a current balancing effect on the six windings is achieved. The winding 1301 is magnetically coupled with the windings 1302 and the winding 1303. The winding 1308 is magnetically coupled with the winding 1309 and the winding 1302. The winding 1307 is magnetically coupled with the winding 1309 as well as with the winding 1303. The six different windings 1301, 1302, 1303, 1307, 1308 and 1309 are embedded in a core 1306. The core 1306 is typically E-shaped. The arrangement is symmetrically with respect to the center lines 1304 and 1305.

FIG. 13 shows a winding arrangement, which represents an alternative to that depicted in FIG. 12. The advantage of this configuration is that the windings 1301, 1302, 1303, 1307, 1308, 1309 leave the area of the core 1306 only at two different places. This leads to advantages with respect to construction and maintenance of the power transformer.

FIG. 14 shows details of the arrangement depicted in FIG. 13. The FIG. 14 shows a part of the transformer 1403. The part of the transformer 1403 could be a part of the primary side of the transformer as well as a part of the secondary side of the transformer. It is shown the magnetically couplement of different galvanically isolated windings 1401, 1402 and 1405. The winding 1401 is magnetically coupled with the winding 1402 with the help of a common magnetic flux. The windings 1402 and the winding 1405 are coupled with a common magnetic flux with the help of overlapping areas, which comprise a magnetic flux. The windings 1401, 1402 and 1405 are embedded in a core 1404. The core 1404 is typically E-shaped.

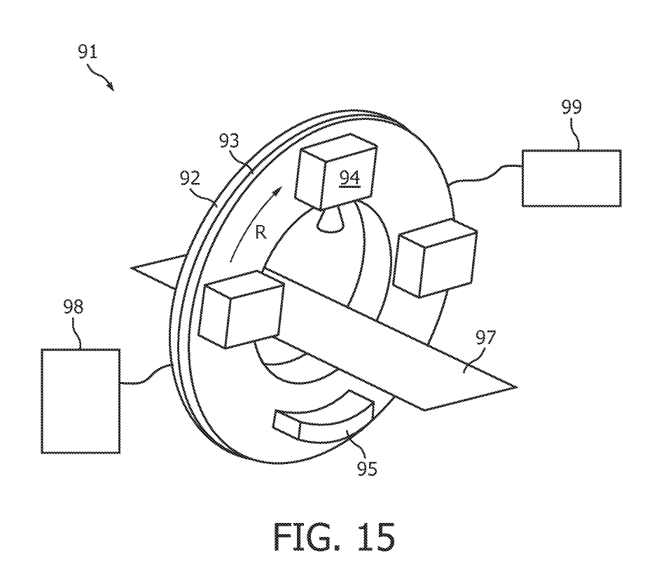

FIG. 15 shows an exemplary embodiment of a computer tomography gantry 91 arrangement. The gantry 91 comprises a stationary part 92 connected to a high frequency power source 98 and a rotary part 93 adapted to rotate relative to the stationary part 92. An X-ray source 94 and an X-ray detector 95 are attached to the rotary part 93 at opposing locations such as to be rotatable around a patient positioned on a table 97. The X-ray detector 95 and the X-ray source 94 are connected to a control and analysing unit 99 adapted to control the X-ray detector 95 and the X-ray source and to evaluate the detection results of the X-ray detector 95.

It should be noted that the term ‘comprising’ does not exclude other elements or steps and the ‘a’ or ‘an’ does not exclude a plurality. Also elements described in association with the different embodiments may be combined.

It should be noted that the reference signs in the claims shall not be construed as limiting the scope of the claims.

### LIST OF REFERENCE SIGNS

91 Computer tomography gantry,

92 Stationary part of the gantry,

93 Rotary part of the gantry,

94 X-ray source,

95 X-ray detector,

97 Table,

98 High frequency power source,

99 Control and analysing unit,

101 Computer tomography gantry,

102 Part of a computer tomography gantry,

103 Stationary part of a transformer,

104 Stationary part of a transformer,

105 Rotary part of a transformer,

106 Inverter,

107 Supply line,

201 Mains input stage,

202 Inverter,

203 Inverter,

204 Supply line,

205 Supply line,

206 Stationary part of a gantry,

207 Core,

208 Primary winding,

209 Primary winding,

210 Primary winding,

211 Primary winding,

301 Mains input stage,

302 Inverter,

303 Inverter,

304 Inverter,

305 Inverter,

306 Primary winding,

307 Primary winding,

308 Primary winding,

309 Primary winding,

310 Part of a computer tomography gantry,

401 Part of a computer tomography gantry,

402 Inverter,

403 Inverter,

404 Mains input stage,

405 Inverter,

406 Inverter,

501 Part of a computer tomography gantry,

502 Mains input stage,

503 Inverter,

504 Inverter,

505 Current balancing choke,

506 Primary winding,

507 Primary winding,

508 Primary winding,

509 Primary winding,

510 Inverter,

511 Inverter,

512 Current balancing choke,

513 Current balancing choke,

519 Inverter,

520 Inverter,

521 Inverter,

522 Inverter,

516 Supply line,

517 Supply line,

518 Mains input stage,

523 Inverter,

524 Current balancing choke,

525 Current balancing choke,

526 Inverter,

527 Inverter,

528 Inverter,

529 Supply line,

530 Mains input stage,

531 Supply line,

532 Inverter,

533 Current balancing choke,

534 Current balancing choke,

535 Inverter,

536 Inverter,

537 Supply line,

538 Mains input stage,

539 Inverter,

540 Supply line,

541 Current balancing choke,

542 Primary winding,

543 Primary winding,

544 Primary winding,

545 Primary winding,

601 Primary winding,

602 Primary winding,

603 Primary winding,

604 Primary winding,

605 Supply line,

606 Supply line,

607 Supply line,

608 Supply line,

609 Cross-section,

610 Core,

611 Cross-section of the primary winding 604,

612 Inverter,

613 Supply line,

614 Inductance of a current balancing choke,

615 Inductance of a current balancing choke,

616 Inductance of a current balancing choke,

617 Inductance of a current balancing choke,

618 Inductance of a current balancing choke,

619 Inductance of a current balancing choke,

701 Power switching unit,

702 Resonant circuit,

703 Current balancing choke,

704 Transformer,

705 Current balancing choke,

706 Transformer,

707 Rectifier,

708 Capacitor,

801 Inverter,

802 Winding,

803 Core,

804 Inverter,

805 Winding,

806 Inverter,

807 Winding,

808 Center line,

809 Center line,

901 Winding,

902 Rectifier,

903 Center line,

904 Core,

905 Center line,

906 Part of a transformer,

1001 Rectifier,

1002 Diode and switch,

1003 Rectifier,

1004 Transformer,

1101 Winding,

1102 Rectifier,

1103 Rectifier,

1104 Winding,

1105 Core,

1106 Center line,

1107 Center line,

1108 Part of a transformer,

1201 Inverter,

1202 Winding,

1203 Inverter,

1204 Winding,

1205 Core,

1206 Inverter,

1207 Winding,

1208 Center line,

1209 Center line,

1210 Inverter,

1211 Winding,

1212 Winding,

1213 Inverter,

1214 Inverter,

1215 Winding,

1216 Part of a transformer,

1301 Winding,

1302 Winding,

1303 Winding,

1304 Center line,

1305 Center line,

1306 Core,

1307 Winding,

1308 Winding,

1309 Winding,

1310 Part of a transformer,

1401 Winding,

1402 Winding,

1403 Part of a transformer,

1404 Core,

1405 Winding.

1. A device for a computer tomography gantry (91) for transferring contactlessly electrical energy from a stationary part of the gantry (92) to a rotary part of the gantry (93),
wherein the device comprises
a first power transformer,
a second power transformer,
wherein the first and the second power transformers are adapted for transferring the electrical energy,
wherein the first power transformer comprises
a first winding (506, 507, 542, 602, 601, 1202, 1301, 1401) out of the group consisting of a first set of primary windings and a first set of secondary windings of the first power transformer,
wherein the second power transformer comprises
a second winding (508, 509, 543, 603, 604, 1204, 1302, 1402) out of the group consisting of a second set of primary windings and a second set of secondary windings of the second power transformer,
wherein
the first set of primary windings and the second set of primary windings being adapted to be mounted on the stationary part of the gantry (92),
wherein
the first set of secondary windings and the second set of secondary windings being adapted to be mounted on the rotary part of the gantry (93),
wherein the device is adapted to balance the currents of the first winding and the second winding.

2. The device according to claim 1, wherein the first winding (506, 507, 542, 602, 601, 1202, 1301, 1401) and the second winding (508, 509, 543, 603, 604, 1204, 1302, 1402) are magnetically coupled in such a way that the device is adapted to balance the currents of the first winding (506, 507, 542, 602, 601, 1202, 1301, 1401) and the second winding (508, 509, 543, 603, 604, 1204, 1302, 1402).

3. The device according to claim 1, further comprising
a current balancing transformer (505, 512, 513, 533, 534, 541, 703, 705), which is arranged in such a way so that being adapted for balancing the currents of the first winding (506, 507, 542, 602, 601, 1202, 1301, 1401) and the second winding (508, 509, 543, 603, 604, 1204, 1302, 1402).

4. The device according to claim 1, wherein
the first winding (506, 507, 542, 602, 601, 1202, 1301, 1401) is a first primary winding of the first power transformer and
the second winding (508, 509, 543, 603, 604, 1204, 1302, 1402) is a second primary winding of the second power transformer, so that the device is adapted for balancing the currents of the first primary winding (506, 507, 542, 602, 601, 1202, 1301, 1401) and the second primary winding (508, 509, 543, 603, 604, 1204, 1302, 1402).

5. The device according to claim 1, wherein
the first winding (1301, 1401) is a first secondary winding of the first power transformer and
the second winding (1302, 1402) is a second secondary winding of the second power transformer, so that the device is adapted for balancing the currents of the first secondary winding and the second secondary winding.

6. The device according to claim 1, wherein the first and the second power transformers are adapted to be operated with currents of a high frequency, such that the power transformers are adapted to transfer energy in a high frequency.

7. The device according to claim 1, further comprising
an inverter (106, 202, 203, 302, 303, 304, 305, 402, 403, 405, 406, 503, 504, 510, 511, 519, 520, 521, 522, 523, 528, 526, 527, 532, 539, 535, 536, 612, 701, 801, 804, 806, 1201, 1203, 1206, 1210, 1213, 1214), wherein the inverter is adapted to be connected with the first and the second power transformer such that the inverter feeds the first and the second power transformer with electrical energy.

8. The device according to claim 1, further comprising
a rectifier (707, 902, 1001, 1002, 1003, 1102, 1103), wherein the rectifier is adapted to be connected with the first and the second power transformer such that the rectifier rectifies the output voltage of the first and the second power transformer.

9. The device according to claim 1, wherein
the device further comprises
a third power transformer,
a fourth power transformer,
wherein the first power transformer is adapted to be supplied by a first inverter (520), wherein the second power transformer is adapted to be supplied by a second inverter (519), wherein the third power transformer is adapted to be supplied by a third inverter (521), wherein the fourth power transformer is adapted to be supplied by a fourth inverter (522), wherein the first inverter (520) is arranged close to the second inverter (519), wherein the third inverter (521) is arranged close to the fourth inverter (522), wherein the first inverter (520) is supplied by a mains input stage (518) via a first supply line, wherein the second inverter (519) is supplied by the mains input stage (518) via a second supply line, wherein the third inverter (521) is supplied by the mains input stage (518) via a third supply line, wherein the fourth inverter (522) is supplied by the mains input stage (518) via a fourth supply line, wherein the first supply line is considerably shorter than the second supply line, wherein the third supply line is considerably shorter than the fourth supply line.

10. The device according to claim 1, wherein a winding out of a group consisting of the first set of primary windings and the first set of secondary windings of the first power transformer and the second set of primary windings and the second set of secondary windings of the second power transformer is arranged in a circular arc.

11. The device according to claim 1, comprising
a first power transformer with a first winding out of a group consisting of the first set of primary windings and the first set of secondary windings,
a second power transformer with a second winding out of a group consisting of the second set of primary windings and the second set of secondary windings,
a third power transformer with a third winding out of a group consisting of the third set of primary windings and the third set of secondary windings,
a fourth power transformer with a fourth winding out of a group consisting of the fourth set of primary windings and the fourth set of secondary windings, wherein the first, the second, the third and the fourth windings are arranged in four circular arcs.

12. The device according to claim 1, comprising
a first power transformer with a first primary winding,
a second power transformer with a second primary winding,
a third power transformer with a third primary winding,
a fourth power transformer with a fourth primary winding,
a first current balancing transformer, wherein a winding of the first current balancing transformer is wound around a part of the first primary winding and around a part of the second primary winding, so that the first current balancing transformer is adapted for balancing the currents of the first and second primary windings,
a second current balancing transformer, wherein a winding of the second current balancing transformer is wound around a part of the second primary winding and around a part of the third primary winding, so that the current balancing transformer is adapted for balancing the currents of the second and third primary windings,
a third current balancing transformer, wherein a winding of the third current balancing transformer is wound around a part of the third primary winding and around a part of the fourth primary winding, so that the current balancing transformer is adapted for balancing the currents of the third and fourth primary windings.

13. The device according to claim 1, comprising
a first power transformer with a first secondary winding,
a second power transformer with a second secondary winding,
a third power transformer with a third secondary winding,
a fourth power transformer with a fourth secondary winding,
a first current balancing transformer, wherein a winding of the first current balancing transformer is wound around a part of the first secondary winding and around a part of the second secondary winding, so that the first current balancing transformer is adapted for balancing the currents of the first and second secondary windings,
a second current balancing transformer, wherein a winding of the second current balancing transformer is wound around a part of the second secondary winding and around a part of the third secondary winding, so that the current balancing transformer is adapted for balancing the currents of the second and third secondary windings, and
a third current balancing transformer, wherein a winding of the third current balancing transformer is wound around a part of the third secondary winding and around a part of the fourth secondary winding, so that the current balancing transformer is adapted for balancing the currents of the third and fourth secondary windings.

14. A computer tomography gantry (91) comprising a device according to claim 1.

15. A method for transferring contactlessly electrical energy from a stationary part of a gantry (92) to a rotary part of a gantry (93), comprising the steps of:
balancing currents with the help of a device according to claim 1.
